(19) United States
(12) Patent Application Publication
(10) Pub. No.: US 2012/0172485 A1
(43) Pub. Date: Jul. 5, 2012

(54) DENTAL COMPOSITIONS WITH TITANIUM DIOXIDE NANOPARTICLES

(75) Inventors: Jirun Sun, Rockville, MD (US); Wen-li Wu, Rockville, MD (US)

(73) Assignee: ADA FOUNDATION, Gaithersburg, MD (US)

(21) Appl. No.: 13/342,243

(22) Filed: Jan. 3, 2012

Related U.S. Application Data
(60) Provisional application No. 61/429,479, filed on Jan. 4, 2011.

(51) Int. Cl. A61K 6/083; C07F 7/28; C08F 4/64; C08F 2/46; B32B 5/16; A61K 6/00; B82Y 5/00; B82Y 99/00; B82Y 30/00; B82Y 40/00

(52) U.S. Cl. 522/182; 428/402; 556/55; 526/90; 523/118; 523/115; 977/773; 977/783; 977/896; 977/919

(57) ABSTRACT

A polymeric composition comprising modified-TiO₂ nanoparticles, with or without a solvent, and polymer precursors; wherein the modified-TiO₂ nanoparticles comprise titanium dioxide nanoparticles modified with a short-chain unsaturated compound comprising 2 to 10 carbon atoms. The polymeric composition may be used in dental compositions such as dental adhesives, dental composites, and dental sealants.

This application claims priority to U.S. Provisional Application Ser. No. 61/429,479, filed Jan. 4, 2011, which application is incorporated by reference in its entirety.

### FIELD OF THE INVENTION

The invention relates to compositions containing titanium dioxide nanoparticles, and in particular to polymeric compositions containing modified-titanium dioxide nanoparticles and use of the polymeric compositions in dental applications such as dental adhesives and dental composites.

### BACKGROUND OF THE INVENTION

Titanium dioxide (TiO₂) particles have at least three superior properties, high refractive index, excellent mechanical properties, and unique photo-catalytic activities; but balancing these properties and optimizing their performance are challenging. The major application of TiO₂ particles is as pigments, utilizing their high refractive index and bright white color, but the high refractive index also causes low transparency, which limits the use of photo-polymerization. Yu et al. (Dental materials, 2009, 1142-1147) found that resin composites with 0.1% by mass to 0.25% by mass of titania particles would simulate the opalescence of human enamel. On the other hand, low transparency of dental resin is a major concern in dentistry. Most dental resin restorations use light to cure the resin. Insufficient light penetration produces incompletely cured resins or non-homogeneously cured resins, which reduces their mechanical performance.

In other applications (U.S. Pat. No. 6,667,360 and U.S. Pat. No. 20100189940), nanosize TiO₂ particles were used as fillers to improve the mechanical properties of polymers. In these applications, a relatively large amount of TiO₂ particles were used to improve the mechanical properties. In examples of U.S. Pat. No. 20100189940, the maximum improvement in modulus was approximately 50% by adding 40% mass fraction (approximately 15% by volume) of TiO₂ particles. Such large amount of TiO₂ particles will inevitably block light transmittance in resins, which impedes the use of TiO₂ particles, especially in dental adhesives and dental composites, where light irradiation is generally used to initiate polymerization.

In another application (Japanese Pat. No. 200533607), the photo-catalytic activities were used in dental adhesives to mask the stain and prevent bacterial attack, but the bond strengths (<11 MPa) of these adhesives were weak. Low bond strength made the adhesive vulnerable to stress caused by shrinking of dental composites during curing (polymerization), which is a common limitation for most of the resin composites. Weak bonds between resin and tooth substrate can also lead to breakdown of restoration from routine oral activities such as mastication and paranormal activities. The broken bonds between tooth and resin composites are highly responsible for gaps and microleakage at the interface of tooth and resin composites, which are suspected reasons for the initiation of secondary caries and restoration failures.

### SUMMARY OF THE INVENTION

The present invention provides improvements to the performance of polymeric compositions including dental compositions, such as dental resin, dental adhesives, dental sealant and dental composites, by adding a significantly small amount of modified-TiO₂ nanoparticles as rigid and multi-functional points. The outstanding mechanical properties and unique photo-catalytic properties of TiO₂ nanoparticles are united together in polymeric compositions, such that the amount of TiO₂ nanoparticles needed to enhance the mechanical properties of polymeric compositions is significantly reduced.

First, the TiO₂ nanoparticles are functionalized with short chain C═C bonds. It is believed that these double bonds on the nanoparticles generate more cross-links and rigid bridges between nanoparticles and resin networks, which is an analogous concept in rubber vulcanization. Furthermore, the modified-TiO₂ nanoparticles produce free radicals upon light irradiation, and it is believed that these free radicals improve the degree of vinyl conversion (DC) of polymer precursors. The successful combination of the increased cross-links and DC and excellent mechanical properties of TiO₂ nanoparticles greatly enhance the modulus and hardness of the dental compositions by adding a small amount of modified-TiO₂ nanoparticles. Surprisingly, using no more than 0.1% by mass or 0.03% by volume of modified-TiO₂ nanoparticles, the modulus of dental resins were improved by approximately 40%, and the shear bond strengths of dental adhesives were increased by 30%. Also beyond expectation, the modified-TiO₂ nanoparticles possess superior photo-catalytic properties comparing to their unmodified counterparts.

The present invention is thus directed to balancing the functions of modified-TiO₂ nanoparticles to achieve the optimal performance of polymeric materials, such as dental compositions, including dental resins, dental adhesives, dental sealant, and dental composites, by adding a significantly low amount of modified-TiO₂ nanoparticles.

In one aspect, the present invention is directed to polymeric compositions comprising the combination of modified-TiO₂ nanoparticles, with or without solvent, and polymer precursors.

In another aspect, the present invention is directed to a method of modifying TiO₂ nanoparticles with short-chain unsaturated compound comprising 2 to 10 carbon atoms with or without branch chains.

In another aspect, the modified-TiO₂ nanoparticles comprise carbon-carbon double bonds physically or chemically attached to the TiO₂ nanoparticles.

In another aspect, the present invention is directed to a method of preparing polymeric compositions comprising mixing the modified-TiO₂ nanoparticles and the solvent with the polymer precursors to form a dispersion or an organosol.

In another aspect, the present invention is directed to a process of forming modified-TiO₂ nanoparticles colloidal dispersions or organosols by centrifuging the mixture of modified-TiO₂ nanoparticles and the solvent.

In another aspect, the present invention is directed to utilize the combination of modified-TiO₂ nanoparticles and polymer precursors in dental adhesive to bond a tooth substrate and dental composite together.

In another aspect, the present invention is directed to utilizing modified-TiO₂ nanoparticles as an initiator/co-initiator of photo-polymerization to improve the degree of vinyl conversion of polymer precursors via light irradiation, preferably blue light irradiation.

In another aspect, the present invention is directed to utilizing modified-TiO₂ nanoparticles as additives and catalysts to polymeric materials, including dental polymeric materials, such as dental resins, dental resin composites, dental adhesive and dental sealant, and synthetic rubbers, epoxy fiber glass, paint and coating materials, to improve the mechanical performance of the polymeric materials including enhancing modulus and hardness.

In another aspect, the present invention is directed to utilizing modified-TiO₂ nanoparticles as additives and catalysts to polymeric materials, including dental polymeric materials, to control/manipulate/modify the hydrophilicity of the polymeric materials by light irradiation and varying the mass fraction of modified-TiO₂ nanoparticles.

In another aspect, the modified-TiO₂ nanoparticles possess enhanced photo-catalytic activities compared to the non-modified TiO₂ nanoparticles. The modified-TiO₂ nanoparticles produce more free radicals upon light irradiation, and the life time of these free radicals is longer.

In another aspect, the present invention is directed to utilizing modified-TiO₂ nanoparticles as additives to polymeric materials, including dental polymeric materials, to kill bacteria and fungi.

### BRIEF DESCRIPTION OF THE DRAWINGS

FIG. 1A) FTIR spectra of acrylic acid and AP25 and the subtraction spectrum of the spectra of AP25 and P25 (inset); FIG. 1B) TGA curves of P25 and AP25; and FIG. 1C) XRD patterns of P25 and AP25.

FIG. 2 TEM images of AP25 nanoparticles at different magnifications

FIG. 3 shows the degree of vinyl conversion (DC) at different mass fractions of modified-TiO₂ (M-TiO₂) nanoparticles in ethylenedimethacrylate (“EDMA”), and in mixtures of bisphenol A glycidyl methacrylate (“BisGMA”), triethyleneglycol dimethacrylate (“TEGDMA”). The mass ratios of BisGMA and TEGDMA are 3:1, 1:1 and 1:3. The error bars represent standard deviation of 50 FTIR measurements.

FIGS. 4A and 4B show nanoindentation results at 500 nm indentation depth and 1500 nm indentation depth: elastic modulus (E) (FIG. 4A) and hardness (H) (FIG. 4B) of resin mixtures containing equal mass ratio of BisGMA and TEGDMA and various mass fractions of M-TiO₂ nanoparticles. The error bars in FIGS. 4A and 4B represent standard deviation of fifteen measurements on one sample.

FIG. 5A Histogram of elastic modulus (E) for a resin mixture (BisGMA and TEGDMA at 1:1 mass ratio) with a mass fraction of 0.08% AP25. The histrogram was constructed from 40 indents using a 1 μm 60° cone indenter in the center of the cross-sectioned sample. FIG. 5B Indents across the interface of the cross-sectioned BisGMA and TEGDMA at 1:1 mass ratio with a mass fraction of 0.08% AP25 and the mounting epoxy. These indents were conducted using a 1 μm 60° cone indenter. The mounting epoxy has the lower modulus.

FIG. 6 Moduli of one M-TiO₂-polymer (made of BisGMA and TEGDMA 1:1 by mass) determined by nanoindentation and film indentation.

FIG. 7. Shear bond strength (SBS) when resins at six mass fractions of AP25 were used as dentine adhesives. Scotchbond (3M ESPE, St. Paul, Minn., USA) was used as the control. The error bars represent standard deviation of 3-5 measurements.

FIGS. 8A and 8B show the EPR spectra of unmodified TiO₂ nanoparticles and M-TiO₂ at different time periods of UV irradiation. FIG. 8C shows the free radical intensity as a function of time during and after UV irradiation.

FIG. 9A shows water contact angle of M-TiO₂-polymer (the polymer precursor was mixture of PMGDM and HEMA at 1:1 mass ratio.) with different mass fractions of M-TiO₂ nanoparticles before light irradiation; and FIG. 9B shows the difference of water contact angle on three polymers and their M-TiO₂-composites before and after light irradiation. The error bars represent standard deviation of three measurements.

### DETAILED DESCRIPTION OF THE INVENTION

Silanization has been successful in dispersing large amounts of silica into polymer precursors, but the same approach does not work well for TiO₂ nanoparticles due to the different physical and chemical surface properties, especially the high refractive index of TiO₂ nanoparticles. Ligand exchange is a versatile and successful method to desirably tune the surface properties of TiO₂ nanoparticles. This method exchanges short chain acids for fatty acids that are used as surfactants and dispersants on the surface of TiO₂ nanoparticles. The process forms hydrophilic TiO₂ nanoparticles from hydrophobic TiO₂ nanoparticles. But this approach involves fatty acids. The fatty acids are surfactants and difficult to remove completely once involved, and may jeopardize the strength of the dental compositions.

In the present invention, commercially available TiO₂ nanoparticles are modified with organic compounds (for example acrylic acid) that contain ethylenically unsaturated functionality to provide modified-TiO₂ (M-TiO₂) nanoparticles. It is believed that the double bond attached on M-TiO₂ provides a rigid connection between the polymer network and the TiO₂ nanoparticles which are locked into the polymer network through conversion of vinyl groups to C—C bonds.

The dispersion method of TiO₂ nanoparticles into polymer precursors is also important. Successful surface modification of TiO₂ nanoparticles will not guarantee the good dispersion of particles in the polymer precursors, but a good dispersion is vital for the best performance of TiO₂-containing polymeric compositions. While there is no quantitative measure of a “good” dispersion, a good dispersion will lack large agglomerates of modified-TiO₂ nanoparticles.

The modified-TiO₂ nanoparticles may be added into polymeric material such as dental resins to improve the performance of the resins by providing a higher degree of vinyl conversion, greater elastic modulus and hardness, and stronger shear bond strength when used as dental adhesive. For example, the performance of studied dental resins was improved dramatically by adding a small amount of modified-TiO₂: 1) the DC of the mixture of BisGMA and TEGDMA was improved by approximately 5% by adding of 0.08% mass fraction of modified-TiO₂; 2) the elastic modulus of the BisGMA and TEGDMA mixture was enhanced by ≈48% by adding 0.06% mass fraction of modified-TiO₂, and its hardness was more than doubled by adding 0.06% (mass fraction) of AP25; 3) the mean SBS was increased approximately 30% when 0.1% mass fraction of modified-TiO₂ was added to the dentin adhesive. In another example, the flexural modulus of EDMA with 0.1 mass % of modified-TiO₂ was more than 20% higher than the EDMA with the same amount of unmodified-TiO₂.

These new modified-TiO₂-polymers can be used in thermoplastic, thermoset or cross-linked polymeric materials, including dental polymeric materials, such as dental resins, dental resin composites, dental adhesive and dental sealant, and synthetic rubbers, epoxy fiber glass, paint and coating materials, anti-microbial packing materials, scratch resistant materials, and self-clean materials.

Modified-TiO₂ nanoparticles can be prepared by modifying titanium dioxide nanoparticles with organic compounds possessing a polymerizable group(s), preferably C—C double bond. The modified-TiO₂ nanoparticles can be prepared by mixing TiO₂ nanoparticles with a solvent and an organic compound with polymerizable groups.

The solvent may be water, an organic solvent with a boiling point below 159° C. including ethanol, methanol, toluene, ethyl ether, cyclohexane, iso-propanol, chloroform, ethyl acetate hexane, heptanes, etc., or mixtures thereof

The organic compounds include acrylic acid, carboxylic acid, amine, phosphonic acid, phosphine, and silanizing agents (for example 3-methacryloxypropyl-trimethoxysilane). Short-chain (2 to 10 carbon atoms) compounds with or without branch chain(s), for example acrylic acid, are preferred. These short-chain compounds provide rigid bonds between modified-TiO₂ nanoparticles and the polymer network.

Mixing or agitation occurs at a temperature in the range of approximately 20° C. to 80° C., typically 20° C. to 40° C., for an amount of time sufficient to form a complex on the surface of TiO₂ nanoparticles thus forming the modified-TiO₂ nanoparticles. After modification, the mass fraction of organic components in the modified-TiO₂ nanoparticles is in the range from 0.5% to 10% determined by thermal gravimetric analysis.

The modified-TiO₂ nanoparticles in the solvent may be centrifuged at a spin rate in a range from 1000 rpm to 5000 rpm for a time from 1 min to 10 min to disperse the M-TiO₂ nanoparticles in the solvent. The centrifuge step is important to obtain uniformly distributed nanoparticles in the solvent. Such dispersion is an organosol or colloidal dispersion of modified-TiO₂ nanoparticles, i.e. particles dispersed in an organic or aqueous solvent. The particles in these organosol or colloidal dispersions have a narrow size distribution and are in a controlled agglomeration. The average hydrodynamic radius (Rₕ) determined by dynamic light scattering of the agglomerates is from 20 nm to 1000 nm. For example, the average Rₕ of modified-TiO₂ agglomerates in ethanol was 267 ±32 nm determined by dynamic light scattering.

The modified-TiO₂ nanoparticles have a particle size or crystalline diameter, greater than 1 nm and less than 100 nm determined by transmission electronic microscopy. The crystal form of the modified-TiO₂ nanoparticles comprises mainly of anatase (>60%) and rutile (<40%). As an example, one modified-TiO₂ used in this application was composed of 77.9% mass fraction of anatase and 22.1% mass fraction of rutile.

The modified-TiO₂ nanoparticle dispersions are combined with polymer precursors to form modified-TiO₂-polymers. The polymer precursors may possess one or more ethylenically unsaturated polymerization functionality, preferably carbon-carbon double bonds. Polymer precursors include but are not limited to derivatives of acrylate, methacrylate and dimathacrylates such as ethylenedimethacrylate (“EDMA”), bisphenol A glycidyl methacrylate (“BisGMA”), triethyleneglycol dimethacrylate (“TEGDMA”), 1,6-bis(methacryloxy-2-ethoxycarbonylamino)-2,4,4-trimethylhexane (UDMA), pyromellitic glycerol dimethacrylate (PMGDM), 2-hydroxyethyl methacrylate (HEMA) and the mixtures thereof The polymer precursors may also be derivatives of one or more of the following function groups: ether, carbonate, silicone, olefin, urethane, styrene, vinylaromatic, amide, imide, vinylhalide, phenylene oxide, ketone, and blends thereof

The polymers after polymerization of the above polymer precursors in the present invention include acrylate resin (polymers and copolymers of acrylate, methacrylate and dimethacrylate esters), epoxy, polycarbonate, silicone, polyester, polyether, polyolefin, synthetic rubber, polyurethane, nylon, polystyrene, polyvinylaromatic, polyamide, polyimide, polybinylhalide, polyphenylene oxide, polyketone and copolymers and blends thereof. Copolymers include both random and block copolymers.

The percentage of polymers in modified-TiO₂-polymers is no less than 20% by mass, and the percentage of modified-TiO₂ nanoparticles is not greater than 5% by mass, preferably 0.005% to 2%, or 0.005% to 0.5%, or 0.005% to 0.2% more preferably 0.05% to 0.1%.

Modified-TiO₂-polymer composites may be made by mixing together polymer precursor(s), initiator(s) for polymerization, an organosol or a colloidal dispersion containing modified-TiO₂ nanoparticles, optionally inorganic fillers, and a solvent. The solvent is removed from the mixture via vacuum for example, at 0.5 to 2 mmHg, 20 to 40° C. The solvent can also be removed by blowing dry air through the mixture. The solvent-free mixture is converted into modified-TiO₂-polymers via heat or light irradiation, typically visible light, and preferably blue light (wavelength is approximately in the range from 350 nm to 550 nm.)

The modified-TiO₂ nanoparticles may function as photo-initiators or co-initiators when combined with the polymer precursors and subjected to light irradiation. The modified-TiO₂ nanoparticles enhance the degree of vinyl conversion (from carbon-carbon double bonds to carbon-carbon single bond) under visible light irradiation of the polymer precursors.

The modified-TiO₂ nanoparticles may have superior photo-catalytic activities than their non-modified counter parts. The photo-catalytic activities are evaluated by the ability to produce free radicals upon light irradiation and the life time of these free radicals. Nanoparticles that produce more free radicals over the same irradiation period are desirable. Long life time of free radicals is also desirable for durable photo-catalytic activities. Comparing to their non-modified counterparts, the modified-TiO₂ nanoparticles generate more free radicals via light irradiation, and these free radicals have a longer life time.

The resulting modified-TiO₂-polymers may function as antimicrobial materials. These modified-TiO₂-polymers kill fungi and bacteria or prevent growth thereof in the presence of light including UV and visible light and also in darkness after being irradiated with UV or visible light. The bacteria include both Gram positive and Gram negative cells. Further, the antimicrobial properties of these materials may be rejuvenated with light irradiation.

Materials made of the modified-TiO₂ nanoparticles-polymers can have tunable hydrophilicity. The hydrophilicity of the material is tunable based on the polymer, the mass fraction of modified-TiO₂ and the duration of light irradiation using UV or visible light. The materials are relatively hydrophobic before light irradiation and become more hydrophilic after light irradiation. The hydrophilicity is determined according to water contact angle measured by goniometer. A bigger water contact angle means relatively more hydrophobic. The materials may go back to be relatively hydrophobic after being kept in dark.

The modified-TiO₂-polymer-precursors may be used in a variety of materials for example, as components in dental adhesives, dental resin composites and dental sealants. A typical dental adhesive contains resin precursors, curing initiators, coinitiators, inhibitors or stabilizers, solvents, and sometimes inorganic fillers and is used to bond tooth substrates and composites. The modified-TiO₂ nanoparticles may perform multiple functions in the dental adhesives systems, including initiator, co-initiators, cross-linking agents, and re-enforcing fillers. A dental adhesive typically comprises 0.05 to 1 mass % modified-TiO₂ nanoparticles and polymer precursors.

Dental adhesives in accordance with the present invention include operation procedures which three steps. Step 1 is acid etching, Step 2 is applying primer, and step 3 is applying bonding agents. The materials of each step are acid, primer, and bonding agent. Type 1 dental adhesives use these three steps and the modified TiO₂-polymer precursor can be used as a bonding agent and/or as part of a primer. In Type 2, step 2 and step 3 are combined, or step 1 and step 2 are combined. In Type 3, all three steps are applied together. The modified TiO₂-polymer precursor can be used in Type 2 and Type 3 where primers and bonding agents are applied.

A typical dental composite consists of a resin-based polymeric matrix, such as a bisphenol A-glycidyl methacrylate (BisGMA) or urethane dimethacrylate (UDMA), and an inorganic filler such as silicon dioxide (silica), and is used for tooth caries restoration. A typical dental sealant is a dental treatment consisting of applying a thin coating of polymeric materials to one or more teeth, for the intended purpose of preventing dental caries (cavities) or other forms of tooth decay.

### EXAMPLES

### Example 1

Unmodified-TiO₂ nanoparticles (P25, AEROXIDE TiO₂, known photo-catalytically active materials composed with both anatase and rutile phases, provided by Evonik) were modified with acrylic acid and the product was labeled as M-TiO₂ (or AP25 in the examples.) A mixture of acrylic acid (7.2 mL) and water (0.8 mL) was first stirred in a 25 mL vial. Then 8 mL ethyl acetate solution of P25 (0.2 g) was added dropwise in 30 min, and then the mixture was agitated at 37° C. for 24 h. A milky solution was formed and transferred into a 50 mL centrifuge tube. The contents of the tube were centrifuged at 3000 rpm for 6 min. AP25 collected in the bottom of the tube as a solid layer. This solid layer was then redistributed in 25 mL of ethanol and centrifuged at 3000 rpm for 3 min to remove the remaining acrylic acid. The same step was also used to prepare AP25 organosol in ethanol. The AP25 organosols (≈0.12% by mass or ≈0.02% by volume) in ethanol did not form precipitate for several days. The mass fraction of AP25 in these solutions was determined as the solid percentage after the ethanol was removed in vacuum oven (1-2 mmHg) at 22° C. for 24 h.

### Example 2

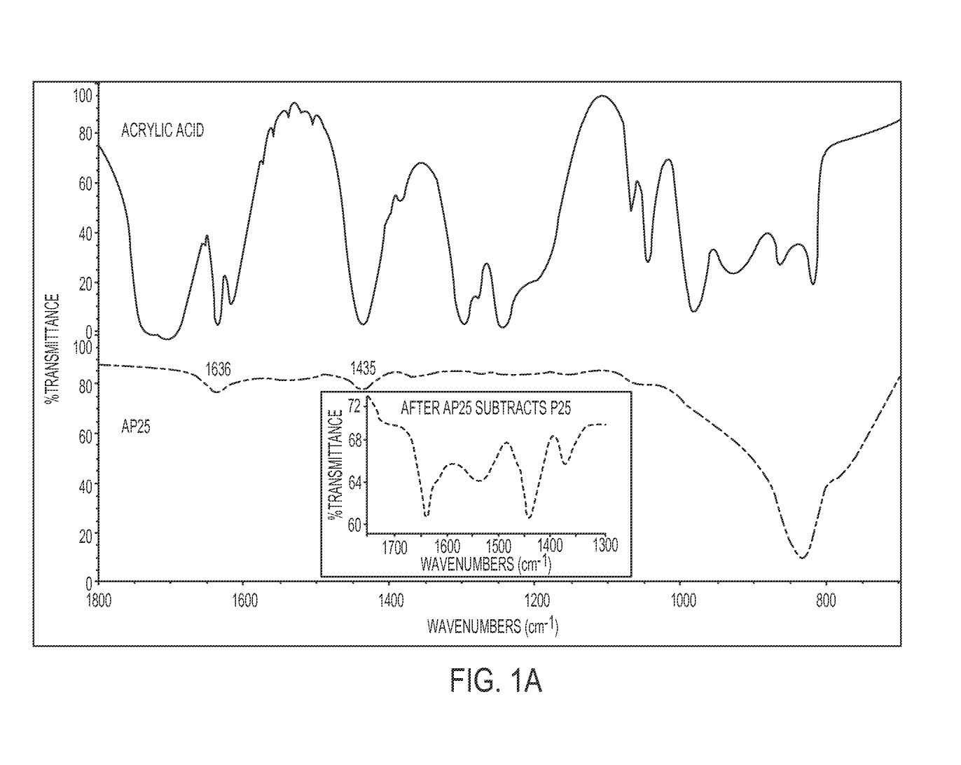

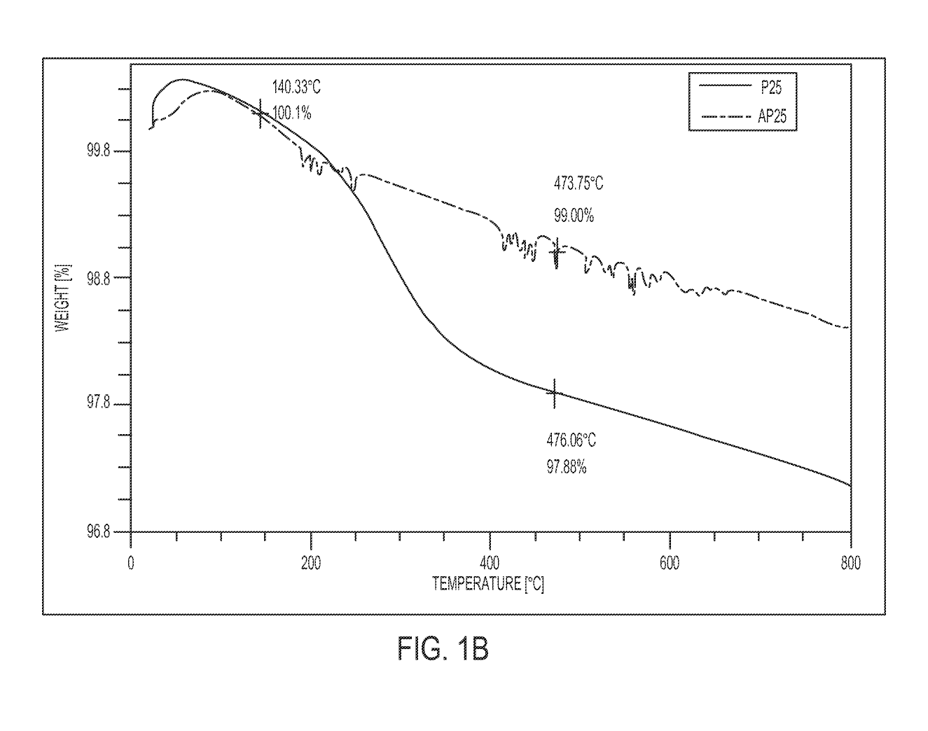

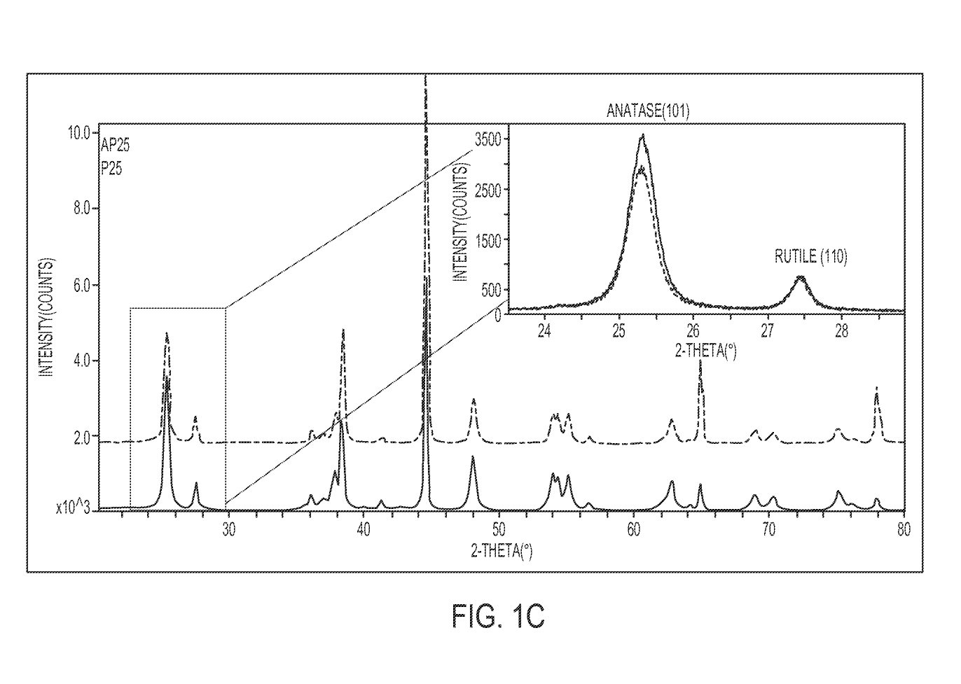

FTIR spectroscopy and thermal gravimetric analysis (TGA) examination confirmed the attachment of the acrylic acid onto the surface of the TiO₂ nanoparticles. The —C═C— signal at 1636 cm⁻¹ in the FTIR spectrum of AP25 nanoparticles (FIG. 1A) verified the existence of the double bond after the surface treatment of TiO₂ nanoparticles. This peak also exists in the subtraction spectrum of the spectra of modified and non-modified TiO₂ nanoparticles (see inset of FIG. 1A), which confirmed that the double bonds were added onto TiO₂. For comparison, the FTIR measurements of the nanoparticles were run under the same conditions. The vibration of free carbonyl group at 1703 cm⁻¹ disappeared, which indicated no residual acrylic acid.

In addition to the FTIR results, TGA results showed 2.08±0.17% of weight loss of M-TiO₂ nanoparticles from 140° C. to 500° C. while 0.95±0.21% of weight loss of P25 was found in this temperature range (FIG. 1B), thus, the attachment of acrylic acid to the surface of TiO₂ Nanoparticles was approximately 1% by mass.

The shape and average size of the P25 did not change after reaction with acrylic acid. The average size of the AP25 and P25 was approximately 25 nm. Theoretically, if the surface of a TiO₂ nanoparticle was covered by a monolayer of acrylic ester, the product after the reaction of acrylic acid and TiO₂, the maximum mass fraction of the organic monolayer will be approximately 4%. This value was based on the assumption: 1) each ester group occupied an area whose length was twice of the length of C═O bond; 2) the ester groups were side by side on the TiO₂ surface. The AP25 has a mass fraction of approximately 1% of organic attachment determined by TGA.

The above results indicated that the double bonds from the acrylic acid were successfully attached to the surface of TiO₂ nanoparticles. These double bonds have the potential to lock the TiO₂ nanoparticles into the polymer network upon photo-polymerization. In return, the AP25 nanoparticles could be creating macro-cross-linked hard points in the network due to the excellent mechanical properties of TiO₂ (E≈230 GPa). Even if two double bonds on a single AP25 nanoparticle react, additional crosslinks will be achieved. Above all, the attachment of double bonds on TiO₂ nanoparticles is essential to achieve strong TiO₂-containing resins.

The AP25 TiO₂ nanoparticles used in this study were mixtures of anatase (101) and rutile (110) phases and dominated by anatase phase. The fraction of the rutile phase (Fᵣ) in the mixtures was evaluated according to X-ray diffraction (XRD) peak intensity using the following formula: Fᵣ=(1+0.8 Iₐ/Iᵣ)⁻¹, where Iₐ and Iᵣ represent the integrated intensities of anatase and rutile diffraction peak. The 2-theta of the peaks used for the intensity calculation of anatase and rutile are at 25.3° and 27.4°, respectively (FIG. 1C). The Fᵣ value of AP25 was 22.1%. The Fᵣ value of P25 was 19.4%.

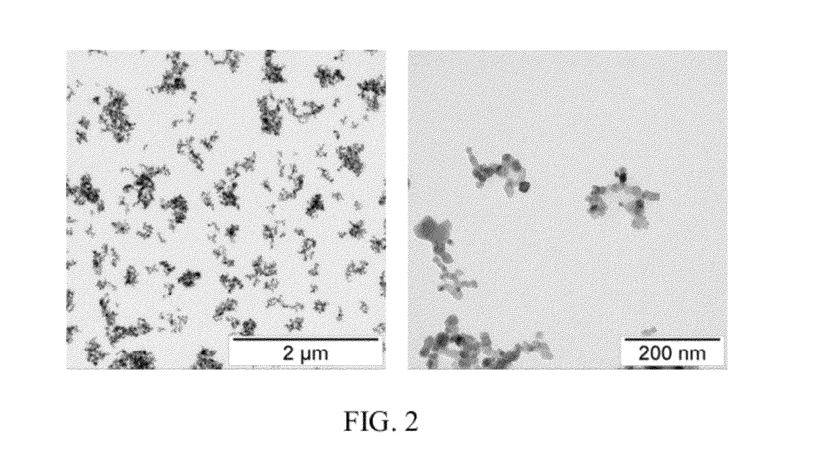

Both DLS and TEM indicated that the majority of the AP25 particles in the ethanol organosols are agglomerates instead of individual nanoparticles. The size of the agglomerates was optimized by using centrifugation to eliminate big agglomerates. TEM images (FIG. 2) shows 1) space among these agglomerates is within 1 μm, which is important to validate the use of nanoindentation for modulus and hardness evaluation; and 2) the size of a single nanoparticle is in the range of 15 nm to 30 nm The hydrodynamic radius (Rₕ) of these agglomerates in an ethanol organosol (Rₕ=267 nm±32 nm) was determined by DLS in 11 angles from 30° to 130° at every 10 degree.

The Rₕ distribution of the AP25 agglomerates in ethanol solution was evaluated by both cumulant and CONTIN methods. The normalized variance (also called the polydispersity parameter), was less than 0.2, which indicated that Rₕ was not broadly distributed. CONTIN was also used to estimate the size distribution. The fit was divided into 30 grid points, and the “dust” term was selected (to handle very large, slow agglomerates). The chosen CONTIN fits for all runs reveal only one major peak which is not less than 90% mass fraction of the amplitude and in a narrow size range. All information supported the notion that the Rₕ of the AP25 agglomerates in ethanol organosols after centrifugation was not distributed over a broad range. The transparency of the organosols was also increased after centrifugation, which was a good indication of evenly distributed small agglomerates.

The narrow size distribution of AP25 agglomerates and the improved transparency of ethanol organosols were firstly due to the good compatibility of AP25 to ethanol, and secondly to the centrifugation process that eliminated the large agglomerates. Large agglomerates were precipitated to the bottom of the centrifuge tube, and the suspended AP25 nanoparticles did not form precipitation for several days. These stable organosols were very important in order to distribute the AP25 nanoparticles evenly into the polymer precursors and vital to achieve a strong polymer with AP25 nanoparticles. Poor distribution of AP25 nanoparticles would diminish the advantage of adding AP25 nanoparticles. Experimental results showed that AP25 nanoparticles were not as effective when dispersed in polymer precursors by sonication as those distributed using ethanol organosols. When the AP25 agglomerates were well distributed into the polymer precursors using AP25 ethanol organosol, the performance of the resins was improved dramatically. Ethanol acted as both solvent and mixing enhancer for even distribution of AP25 in the monomers. In the following examples, degree of vinyl conversion, elastic modulus, hardness and shear bond strength were used to illustrate the performance improvement of dental compositions after adding AP25 TiO₂ₙNanoparticles. The EDMA, BisGMA and TEGDMA were chosen as the base polymer precursors because they are commonly used in the dental field, and their properties have been well studied.

### Example 3

M-TiO₂ nanoparticles can be used as initiator or co-initiators that utilize visible light to initiate polymerization wherein the degree of vinyl conversion (DC) can be improved by more than 20%.

The effectiveness of the M-TiO₂ nanoparticles as co-initiator for photo-polymerization of polymer precursors was correlated with the amount of the nanoparticles added and the chemical or physical properties of the precursors. M-TiO₂ nanoparticles were combined with polymer precursors, including ethylenedimethacrylate (“EDMA”), mixture of bisphenol A glycidyl methacrylate (“BisGMA”) and tetraethyleneglycol dimethacrylate (“TEGDMA”) and mixture of pyromellitic glycerol dimethacrylate (PMGDM), 2-hydroxyethyl methacrylate (HEMA). The activity of M-TiO₂ nanoparticles as co-initiator was determined by calculating the DC using FTIR of the M-TiO₂-polymer-precursors which were cured under visible light.

The most hydrophobic precursor, EDMA, showed the greatest response to the addition of M-TiO₂ nanoparticles. Without M-TiO₂, the DC of EDMA was 69.7±5.4%; the DC of EDMA increased as more nanoparticles were added and reached to a plateau at approximately 92.5% when the concentration of M-TiO₂ nanoparticles was approximately 0.2%. The DC of EDMA was improved more than 20% by adding as little as 0.2% M-TiO₂ nanoparticles.

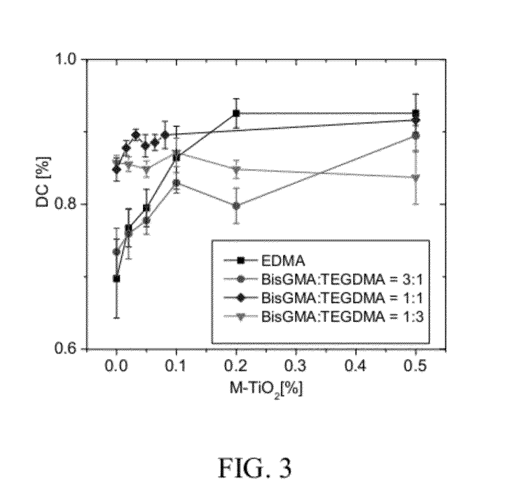

The M-TiO₂ nanoparticles were effective in enhancing DC of polymer precursors. By varying the compositions of BisGMA and TEGDMA, the viscosity of the polymer precursors was adjusted. BisGMA contained rigid phenyl groups and provided strong mechanical properties. TEGDMA is normally used as a diluting precursor to improve processability and DC. As illustrated in FIG. 3, the more TEGDMA in the mixture monomers the higher the DC. The addition of M-TiO₂ nanoparticles modified this trend. The DC of BisGMA-rich precursors was increased approximately by 16% to a value of 89.5±2.2%, while the DC of the TEGDMA-rich precursors remained at approximately 85% for all of the M-TiO₂ nanoparticles concentrations studied.

The M-TiO₂ nanoparticles themselves also initiated the polymerization, but in a very slow rate. When no camphorquinone (CQ) and ethyl 4-N,N-dimethylaminobenzoate (4E) were added, a mixture of BisGMA and TEGDMA (1:1 by mass) that contained 0.5% (by mass) M-TiO₂ nanoparticles turned into soft gel after 2 min blue light irradiation. The DC of the gel was 15.8±6.4%.

It is uncertain whether or not the free radicals initiate the polymerization through the

C=C groups attached on the surface of the TiO₂. If one percent mass fraction of a 25 nm M-TiO₂ nanoparticles is covered by a monolayer of ester groups, approximately 31,000 C=C groups are attached on the surface. When a small portion of these double bonds are utilized to form cross-links in the resin network, the function of M-TiO₂ nanoparticles within the network is similar to that of sulfur in the vulcanization process through which the superior mechanical properties of rubbers are achieved.

The light source (blue light, wavelength 350-550 nm) was used for photo-polymerization. In dentistry, blue light is commonly used in curing dental adhesives and dental resin composites because it is safer than UV and is more powerful than other visible wavelengths. The M-TiO₂ nanoparticles showed obvious improvement on DC of polymer precursor under blue light irradiation.

### Example 4

M-TiO₂-polymers have a significantly higher elastic modulus and hardness than those of TiO₂-free polymers.

Nanoindentation measurements were performed using an Agilent NanoXP instrument equipped with a 10 μm radius, 90° diamond cone indenter. Samples were indented to a maximum depth of 500 nm or 1500 nm using a single loading and the continuous stiffness method. The contact stiffness between the sample and tip was measured by superposing a small oscillation (45 Hz, 5 nm) over the load profile. The loading time was approximately 120 s with a 30 s hold at the maximum load before unloading. This stiffness was used to calculate the elastic modulus of the sample assuming a constant Poisson's ratio of 0.45, a representative value for dental composites. The elastic modulus and hardness were determined as the average value obtained over a depth ranging from 250 nm to 450 nm at a maximum depth of 500 nm and 950 nm to 1450 nm at a maximum depth of 1500 nm for each indent and the average of 15 measurements are reported. All indentation experiments were conducted using a constant indentation strain rate of 0.05 s⁻¹.

Nanoindentation is often used to measure the modulus and hardness of small volumes near the surface of materials. In order to determine whether the mechanical properties of the composites varied with depth and reduced the viscoelastic effects on the indentation measurement, two different maximum depths were investigated using the continuous stiffness measurement (CSM) method. This method collects stiffness data as a function of depth by superposing a sinusoidal oscillation over the loading profile. The stiffness is used in the Hertz contact equations to calculate the elastic modulus of the materials and the hardness is the stress beneath the indenter. The contact area is determined as a function of distance using indentations into a standard fused silica material.

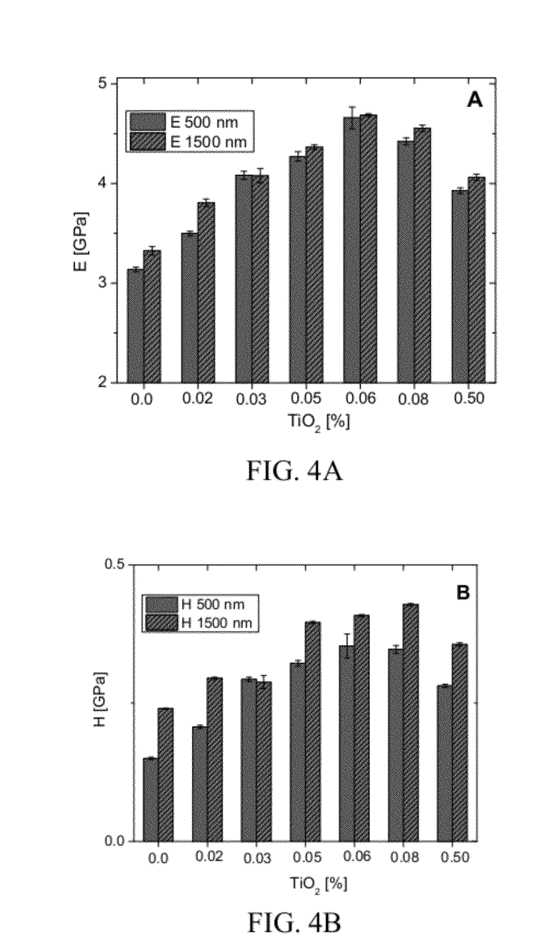

FIG. 4 shows the elastic modulus (FIG. 4A) and hardness (FIG. 4B) of disks with an increasing mass fraction of M-TiO₂ nanoparticles. According to the results from nanoindentation, the elastic modulus and hardness of the resin are improved by adding M-TiO₂ nanoparticles, but there is a maximum increase in properties. The elastic modulus exhibits a sharp increase, 48% with increasing mass fraction of M-TiO₂ nanoparticles up to 0.06% mass fraction at a maximum indentation depth of 500 nm At a maximum indentation depth of 1500 nm, a similar increase, 41%, in elastic modulus is observed at the 0.06% mass fraction. At the highest mass fraction, the elastic modulus decreases which is expected for a material that appears cloudy (low transparency) due to agglomerated particles. Overall, the elastic modulus for the M-TiO₂-polymers measured at two different indentation depths agree fairly well.

Similar behavior is observed for the hardness measurement, although the hardness approximately doubled for the two indentation depths. The maximum hardness was reached when 0.06% mass fraction of M-TiO₂ nanoparticles was added, and it was 2.4 times higher than that of the pure resin. In general, the hardness for the 1500 nm maximum indentation depth was greater than that for the 500 nm maximum indentation depth. Since the elastic modulus indicated no significant differences in mechanical properties with depth, this could be an indication of slight errors (≈10%) in the contact radius at the larger depth.

An optimal mass fraction of M-TiO₂ nanoparticles existed that permitted the polymer to reach its maximum elastic modulus and hardness, which depended on how evenly the M-TiO₂ nanoparticles were distributed within the polymer and the quantity of M-TiO₂ nanoparticles added into the polymer. TiO₂ has a very high hardness and modulus, which contributed to the improvement of mechanical properties. On the other hand, adding unevenly distributed M-TiO₂ nanoparticles will increase the opacity of resins and may cause weak points in the polymer, thus diminishes the advantage gained from adding M-TiO₂ nanoparticles. As a result, the elastic modulus and hardness did not increase proportionally to the mass fraction of M-TiO₂ nanoparticles, but instead those properties began to decrease after reaching the maximum value. Both elastic modulus and hardness testing indicated that the optimal mass fraction of this specific M-TiO₂ nanoparticles was in the range of 0.06% to 0.1%. The density of titanium dioxide is 4.23 g/cm³, and the corresponding volume fraction of M-TiO₂ nanoparticles was 0.014% to 0.021%, which was very close to the volume fraction of ethanol solution of M-TiO₂ nanoparticles after centrifugation.

### Example 5

One formulation (BisGMA and TEGDMA at 1:1 mass ratio with a mass fraction of 0.08% of AP25) was further analyzed using indentation with different indenter geometry (1 μm 60° cone indenter). The sample was cut in half, mounted in cross-section, and mechanically polished to provide a smooth surface to indent. Indents were conducted across the interface between the mounting epoxy and the surface of the composite and the middle of the cross-sectioned samples. These measurements were conducted to determine whether any mechanical property gradients existed through the thickness of the sample.

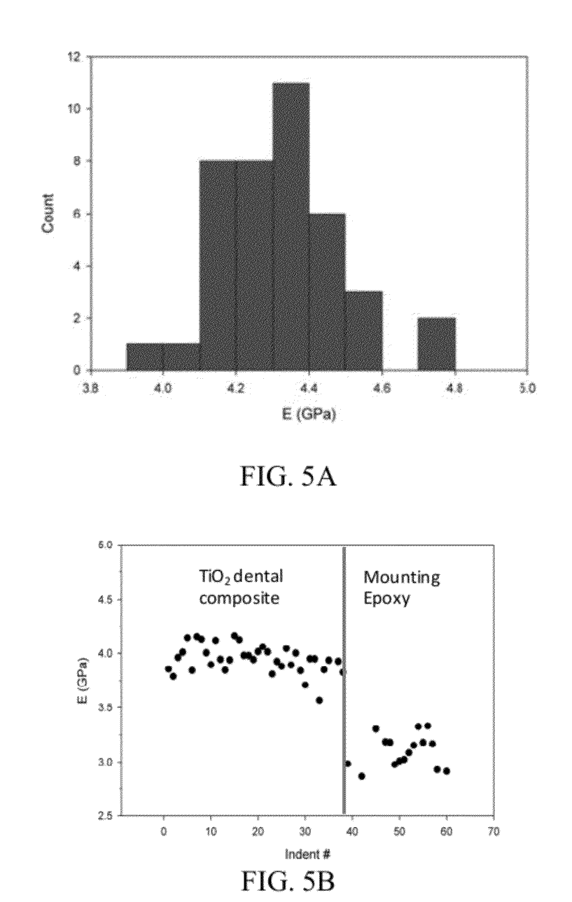

FIG. 5A shows a histogram of 40 indents on the BisGMA and TEGDMA at 1:1 mass ratio with a mass fraction of 0.08% of AP25. The data reflects a normal distribution with a 3.6% covariance and agrees with the data in FIG. 4A. This demonstrates the uniformity of mechanical properties within the bulk of the composite sample. Given the expected size of agglomerates (˜200 nm) in this system, it is not expected to measure individual TiO₂ particles. FIG. 5B shows the elastic modulus measured from within a few micrometers of the surface edge and into the mounting epoxy (lower elastic modulus). The modulus measured here was slightly lower (<10%) than measured previously. Overall, the modulus remains constant, exhibiting a slight decrease near the interface due to the presence of the composite edge and mounting epoxy

### Example 6

Moduli determined using three methods: nanoindentation, microindentation, and 3-point bending. Polymer precursor for this example was mixture of BisGMA and TEGDMA at 1:1 mass ratio.

A microindentation technique using static load indenters was also used to measure the elastic modulus of the AP25 nanocomposites. The gravity load from stainless steel spheres (radius=6.35 mm) indent the substrate, forming a contact area dictated by the substrate modulus and indenter geometry. Contact areas were measured using an inverted optical microscope images (Leica DMIRE II), and image analysis was performed to find contact radii. From Hertzian contact mechanics, the Young's modulus, E, of the substrate could be calculated from indenter geometry and the indentation load, using the previously set Poisson's ratio of 0.45. Indentations were performed at five different positions, and images were immediately taken after the indenter was placed on the polymer substrate. For the 0.5% by mass nanoparticle sample, a thinner sample was required to visualize the indentation due to particle light scattering obscuring the contact area. For this sample, a modified version of the Hertzian indentation model was used to correct for the non-infinite substrate thickness and calculate the elastic modulus.

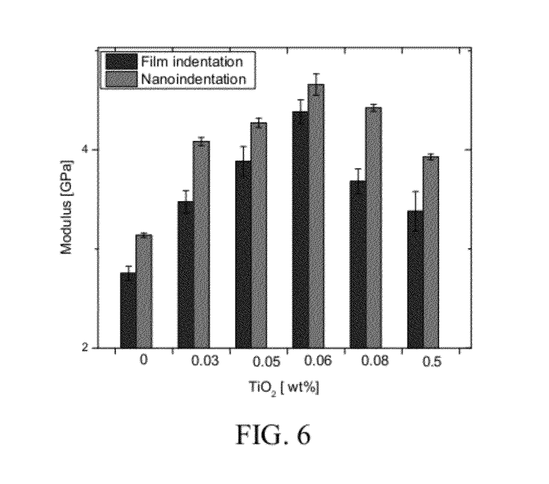

FIG. 6 lists moduli determined using two methods: nanoindentation and microindentation. As mentioned in Example 4, the increase in elastic modulus measured by nanoindentation is greater than that predicted by micromechanics. For comparison, the elastic modulus was also measured by microindentation (thin film indentation), which used a much larger contact area and sample volume to measure the elastic modulus. Microindentation used a larger indenter (12 7 mm in diameter), which increased the contact area (The diameter of contacting area is up to 100 μm.) and measured the average elastic modulus of a larger substrate volume than for nanoindentation. While both measure elastic modulus, nanoindentation is limited sampling polymer near the surface, while the microindentation measurement penetrates deeper into the bulk substrate. In addition, 3-point bending tests were used to measure the flexural modulus of resins. The two indentation methods measured the moduli using contacting surface area from 1 μm up to 100 μm in diameter, and they agree well with each other in terms of the trend of the elastic modulus as a function of mass fraction of M-TiO₂ nanoparticles, but microindentation shows a lower modulus than nanoindentation. This difference could be due to the sampling volumes, if the surface has a slightly higher conversion than the overall bulk. The trend of elastic modulus as a function of mass fraction of M-TiO₂ nanoparticles is also in good agreement with the trend of hardness determined by both nanoindentation and Knoop indentation. In this system, acrylic acid double bonds attached to M-TiO₂ nanoparticles would provide a similar crosslink between the nanoparticles and resin.

The great improvement of modulus and hardness are more likely benefit from the excellent mechanical properties and unique photoactivities of TiO₂ nanoparticles. In addition, the attached double bonds on M-TiO₂ nanoparticles ensure that the nanoparticles are locked into the resin network. In return, the M-TiO₂ nanoparticles created micro-crosslinked hard points in the network. The M-TiO₂ nanoparticles could also generate more crosslinks. Even if two acrylates on a single M-TiO₂ nanoparticle react, additional crosslinks are produced. The higher the DC reaches, the more crosslinks will form. In this case, the M-TiO₂ nanoparticles are not simple additives to the polymers, they make a big difference in terms of the number of crosslinks and polymer chain mobility.

### Example 7

Adding M-TiO₂ nanoparticles increased the flexural modulus more than adding non-modified TiO₂ nanoparticles.

Flexural modulus was determined according to ISO4049: 2009. Five rectangular specimens of each material for each test were made by pipetting the material into a glass tube with a rectangular opening (inside dimension: 25 mm×2 mm×2 mm) Air bubbles were removed by centrifugation, and the tubes were sealed with wax to prevent air-inhibited layers. In this test, all of the bars were cured using a Dentsply Triad 2000 visible light curing unit with a tungsten halogen light bulb (250 W and 120 V) for 2 min each of two opposite sides. After curing, the specimens were stored at room temperature for 24 h. Flexural modulus of the polymers or composites was determined using Universal Testing Machine (Instron 5500R, Instron Corp., Canton, Mass., USA) at a cross-head speed of 1 mm/min. The specimens were placed on a 3-point bending test device, which was constructed with 20 mm distance between supports and ensuring an equally distributed load. The flexural modulus of each polymer or composite was calculated according to ISO4049: 2009.

The flexural modulus (E) of EDMA, EDMA with unmodified TiO₂ nanoparticles (at a mass fraction of 0.1%) and EDMA with M-TiO₂ nanoparticles (at mass fractions of 0.02%, 0.1% and 0.5%) was evaluated using 3-point bending test. The results are listed in Table 1. By adding unmodified TiO₂ nanoparticles, the flexural modulus of EDMA was increased by 12.9%. This improvement much less than those achieved by adding M-TiO₂ nanoparticles. Even with the smallest mass fraction (0.02%) of M-TiO₂, the flexural modulus was enhanced by 34. 9%. This result confirmed that locking TiO₂ nanoparticles into the polymer network made huge difference in mechanical performance of polymers. The flexural modulus as a function of mass fraction agreed well with the results from nanoindentation and microindentation.

TABLE 1
Flexural modulus determined by 3-point bending test

| | | | Improvement |
|---|---|---|---|
| | E (GPa) | STDEV | (%) |
| EDMA | 1.48 | 0.13 | 0 |
| EDMA + 0.1% P25 | 1.67 | 0.03 | 12.93 |
| EDMA + 0.02% M-TiO2 | 1.99 | 0.11 | 34.89 |
| EDMA + 0.1% M-TiO2 | 2.09 | 0.16 | 41.58 |
| EDMA + 0.5% M-TiO2 | 2.03 | 0.02 | 37.88 |

### Example 8

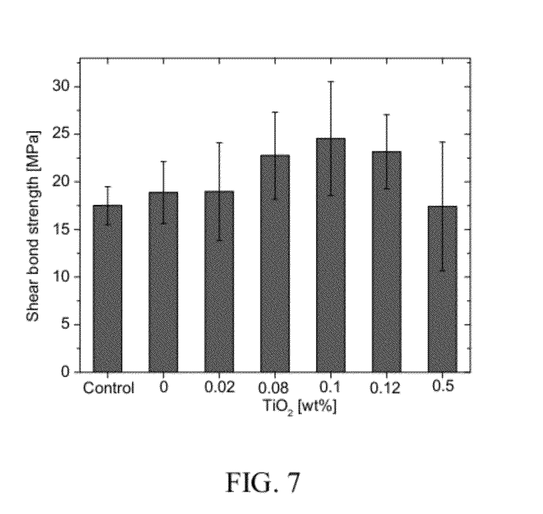

In order to demonstrate clinical applications of these M-TiO₂-resins, the M-TiO₂-polymers were used as dental adhesives to bond tooth substrates and composites. The dental adhesives were generally a 50 μm layer of polymers between composites and dentin. A gold-standard three-step bonding procedure (acid etching, priming, and dental adhesives application) was used to adhere the polymer composite to the ground dentin surface. In order to optimize the number of samples, the polymer precursors (mixture of BisGMA and TEGDMA at mass ration of 1:1) were applied only in the third step as dental adhesives. Total six mass fractions of M-TiO₂ (0%, 0.02%, 0.08%, 0.10%, 0.12%, and 0.50%) were tested. The shear bond strength (SBS) results are shown in FIG. 7. The resins with 0.02% and 0.5% mass fractions of M-TiO₂ have the same mean SBS value as that of the pure resin. The rest compositions generated higher SBS than the TiO₂-free resin. The 0.1%-M-TiO₂-resin has the maximum mean SBS value (24.5 MPa±6.0 MPa) that is approximately 30% higher than that of the pure resin. As expected, a larger amount of M-TiO₂ nanoparticles in the dental adhesive did not produce stronger SBS due to the agglomeration of nanoparticles and increased opacity. When applying the polymer precursor with 0.5% mass fraction of M-TiO₂ nanoparticles as a dental adhesive, it was hard to make smooth surface due to its high viscosity and large amount of particles. Large agglomerates of nanoparticles in the 0.5%-M-TiO₂-resin monomers formed precipitates in two days, while no precipitates were found in the 0.1%-M-TiO₂-resin monomers after 3 months. This indicates a good dispersion of M-TiO₂ nanoparticles in the monomers at the mass fraction of 0.1%.

Further, the mean SBS of dental adhesive was also improved when M-TiO₂ nanoparticles were added into other polymer precursors, including mixture of BisGMA and HEMA, PMGDM and HEMA. The mean SBS was increased 15.8% and 26.5%, respectively, by adding 0.1% of mass fraction of M-TiO₂ nanoparticles into these polymer precursors. It was significantly higher than the SBS of the commercial dental adhesive (Adper Scotchbond multi-purpose adhesives by 3M ESPE).

The SBS test is a simple, often-used screening test for the dental adhesives. The experimental dental adhesives in this study were subjected to loads causing adhesive failure at the dentin-adhesive interface or an occasional cohesive failure in the dentin. The debonding loads of the composite from the dentin surface were in the range of 100 N to 450 N. Under such high loads, the SBS of M-TiO₂-resin adhesives were equal or even 30% greater than that of nanoparticle-free resin adhesive and the commercial control. When dentin adhesives are used in conjunction with composite restorative materials to restore teeth in clinical practice, the restorations, are subjected to loads generated by biting, grinding and chewing that are generally lower than the maximum loads. As dentin adhesives, the M-TiO₂-resins have not only higher (or equal) SBS but also greater elastic modulus and hardness than the nanoparticle-free resins. The laboratory performance of M-TiO₂-resin demonstrated in this study could lead to improved dental adhesives for clinical applications.

### Example 9

The enhanced photo-activities of M-TiO₂ nanoparticles were evaluated by electron paramagnetic resonance spectroscopy (EPR).

EPR measurements were carried out on a BRUKER BioSpin ElexSys500 spectrometer, using a square cavity (TE011 mode) operating under identical parameter settings: microwave frequency 9.38 GHz, field modulation=100 kHz, and microwave power=10 mW. Each sample was placed in a quartz EPR tube in the EPR cavity. A 500 W Xe Arc lamp was used as the UV source for in-situ irradiation experiments. All spectra were obtained at 77 K by sweeping the static magnetic field and recording the first derivative of the absorption spectrum. Unexposed samples and empty EPR tubes were tested, respectively, for reference spectra. A weak pitch standard sample was measured under identical signals and double integrals of EPR spectra were analyzed to quantify the amount of free radicals.

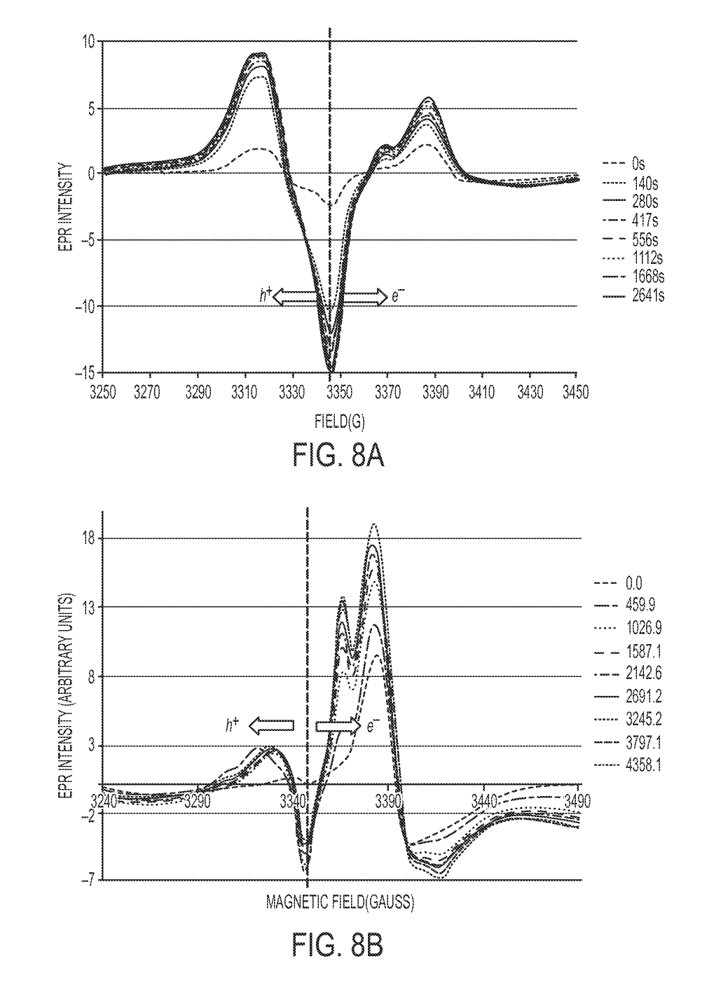

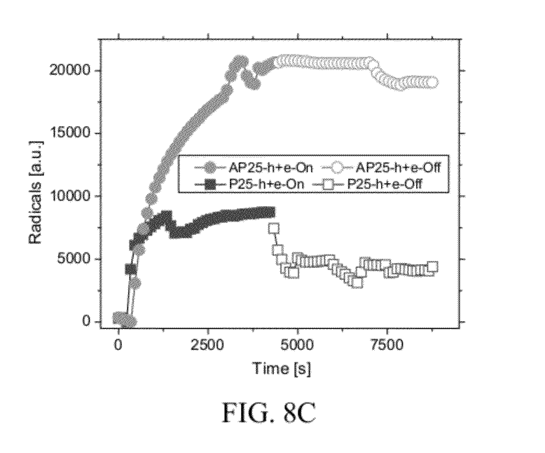

FIGS. 8A and 8B show the EPR spectra of unmodified TiO₂ nanoparticles and M-TiO₂ nanoparticles, respectively, under different periods of UV irradiation. The unmodified TiO₂ nanoparticles produce a slight higher intensity of holes than electrons. Surprisingly, M-TiO₂ nanoparticles significantly increase the production of electron, which is a positive sign for enhancing the photo-activities. The photo-catalytic activities of TiO₂ nanoparticles start from absorbing energy from light and generate electron-hole pairs. Through these electrons and holes, water and oxygen may be converted into powerful oxidation agents, superoxide and hydroxyl free radicals (HO.). These free radicals have the functions including initiating polymerization, modifying hydrophilicity, and decomposing bacteria. When electrons reunited with holes the energy converted from UV irradiation is wasted, unbalanced intensity of electrons and holes improves the efficiency of utilizing energy from light. In order to quantify the free radical production and determine the life time of the free radicals, FIG. 8C shows the free radical intensity as a function of time during and after UV irradiation. Overall, these two nanoparticles all generated free radicals upon UV irradiation, but the free radicals behaved differently on them. For unmodified TiO₂ nanoparticles, the production and consumption of free radicals was equalized after approximately 19 min UV irradiation. In this period, the rate of free radical intensity changed from 37/s to zero. And then, the intensity of free radicals was stabilized around≈8000, where the UV irradiation no longer increased the free radical intensity. Immediately after the UV irradiation was stopped, the free radical intensity dropped and then plateaued after approximately 9 min. The immediate drop of free radical intensity determined the life time of free radicals on unmodified TiO₂ nanoparticles is shorter than 2 min because the interval of the measurement was 110 s. Compared to the unmodified TiO₂ nanoparticles, the M-TiO₂ nanoparticles produced more free radicals in a relatively faster rate and prolonged time period. The free radical intensity increased from zero to approximately 13000 after 19 min UV irradiation, and it was still increasing at approximately 5 /s. The production of free radicals continued until the UV irradiation was stopped at time point 66.9 min. The free radical intensity did not drop and was kept at approximately 20000 for 46 min. The life time of the free radicals on M-TiO₂ nanoparticles was improved more than 23 times. The enhanced free radical production and free radical life time verified the superior photo-catalytic properties of the M-TiO₂ nanoparticles.

### Example 10

The M-TiO₂-polymers have a hydrophilicity that can be manipulated via light irradiation and M-TiO₂ nanoparticles content.

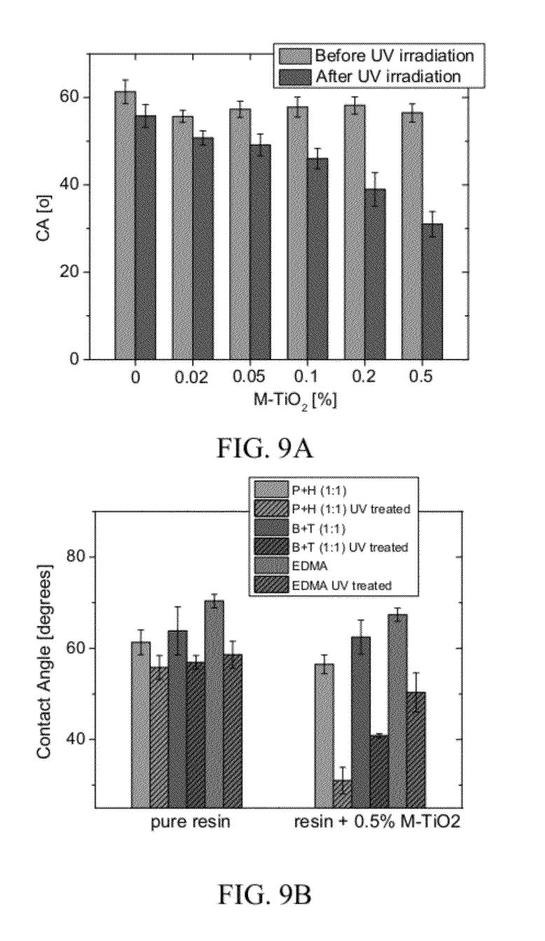

The hydrophilicity was determined using water contact angle measurement. The smaller the water contact angle is the more hydrophilic the material is. FIG. 9A shows the water contact angle of polymer and M-TiO₂-polymers before and after UV irradiation for 20 min. The polymer precursor was a mixture of PMGDM and HEMA (mass ratio 1:1). Five mass fractions (0.01%, 0.05%, 0.1%, 0.2% and 0.5%) of M-TiO₂ were evaluated. Before UV irradiation, that contact angles of all of the M-TiO₂-polymers were the same, and slightly lower than that of the polymer. After UV irradiation, the contact angles decreased. Large amount of M-TiO₂ produced bigger contact angle drop, and the corresponding surface was more hydrophilic. For the polymer with the highest mass fraction of M-TiO₂, its water contact angle changed from 56±2 degree to 31±3 degree.

FIG. 9B illustrates the effects of UV irradiation on three polymers and the corresponding M-TiO₂-polymers (at a mass fraction of 0.5%). The precursors of these three polymers were EDMA, a mixture BisGMA and TEGDMA (1:1 by mass) and a mixture of PMGDM and HEMA (1:1 by mass). Before UV irradiation, the contact angles of these three polymers were in the follow sequence from big to small: EDMA>BisGMA and TEGDMA>PMGDM and HEMA. After UV irradiation, the contact angles of these three polymers were almost the same. The contact angle of Poly-EDMA dropped 12 degree, and the other two polymer dropped 7 degree and 6 degree, respectively. For the corresponding M-TiO₂-polymers, the sequence of contact angle did not change before and after UV irradiation, but the contact angles of each M-TiO₂-polymer decreased approximately 25 degree.

In summary, the performance of polymer was improved dramatically by adding a small amount of M-TiO₂ nanoparticles.
1) the DC of EDMA was improved approximately 22% with addition of 0.2% mass fraction of M-TiO₂nanoparticles;
2) the modulus and hardness of BisGMA and TEGDMA mixture was enhanced up to approximately 48% by adding 0.06% mass fraction of M-TiO₂ nanoparticles;
3) the mean SBS was increased approximately 30% when dentine adhesives were made of M-TiO₂-polymers;
4) the modification of TiO₂ nanoparticles enhanced the photo-catalytic activities by improving the production of free radicals and increasing the life time of free radical; and
5) the hydrophilicity of the M-TiO₂-polymers can be controlled by manipulating UV irradiation and the mass fraction of M-TiO₂.

The effectiveness of the M-TiO₂ nanoparticles is related to the distribution of the nanoparticles in polymer precursors. The optimal concentration of M-TiO₂ nanoparticles for the best performance of polymers appeared to correlate with the maximum concentration of M-TiO₂ nanoparticles without forming large agglomerates.

Above all, by modifying the TiO₂ nanoparticles with short-chain unsaturated compound, the titania's strong mechanical properties and unique photoactive properties were optimized in polymer, which significantly reduced the amount of nanoparticles needed to enhance performance of polymers. Consequently, low mass fraction of TiO₂ nanoparticles overcame the high opacity problem caused by agglomerating and high refractive index of TiO₂ nanoparticles. Thus, the M-TiO₂ nanoparticles may be used to provide strong and long lasting dental composition including dental adhesives and dental composites.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

We claim:

1. Modified-TiO₂ nanoparticles comprising titanium dioxide nanoparticles modified with at least one short-chain unsaturated organic compound comprising 2 to 10 carbon atoms with or without branch chain(s); wherein the mass fraction of organic components in the modified-TiO₂ nanoparticles is in the range from 0.5% to 10% determined by thermal gravimetric analysis.

2. The modified-TiO₂ nanoparticles of claim 1 wherein the short-chain unsaturated organic compound has one or more polymerizable carbon-carbon double bonds.

3. The modified-TiO₂ nanoparticles of claim 1 wherein the at least one organic compound is physically or chemically attached to the TiO₂ nanoparticles.

4. The modified-TiO₂ nanoparticles of claim 1 wherein the modified-TiO₂ nanoparticles are formed by combining unmodified-TiO₂ nanoparticles with at least one short-chain unsaturated organic compound selected from the group consisting of acrylic acid, carboxylic acid, amine, phosphonic acid, phosphine, and silanizing agents.

5. The modified-TiO₂ nanoparticles of claim 1 wherein the modified-TiO₂ nanoparticles have a particle size greater than 1 nm and less than 100 nm

6. The modified-TiO₂ nanoparticles of claim 1 wherein the modified-TiO₂ produce more free radicals upon light irradiation than unmodified-TiO₂ nanoparticles, and the free radicals have a longer life than free radicals produced by unmodified-TiO₂ nanoparticles.

7. The modified-TiO₂ nanoparticles of claim 1 wherein the modified-TiO₂ nanoparticles comprises >60% anatase and <40% rutile.

8. A polymeric composition comprising modified-TiO₂ nanoparticles, with or without a solvent, and polymer precursors; wherein the modified-TiO₂ nanoparticles comprise titanium dioxide nanoparticles modified with at least one short-chain unsaturated organic compound comprising 2 to 10 carbon atoms with or without branch chains; wherein the mass fraction of organic components in the modified-TiO₂ nanoparticles is in the range from 0.5% to 10% determined by thermal gravimetric analysis.

9. The polymeric composition of claim 8 wherein the short-chain unsaturated organic compound has one or more polymerizable carbon-carbon double bonds.

10. The polymeric composition of claim 8 wherein the short-chain unsaturated organic compound is physically or chemically attached to the TiO₂ nanoparticles.

11. The polymeric composition of claim 8 wherein the modified-TiO₂ nanoparticles are formed by combining unmodified TiO₂ nanoparticles with at least one short-chain unsaturated organic compound selected from the group consisting of acrylic acid, carboxylic acid, amine, phosphonic acid, phosphine, and silanizing agents.

12. The polymeric composition of claim 8 wherein the modified-TiO₂ nanoparticles have a particle size greater than 1 nm and less than 100 nm

13. The polymeric composition of claim 8 wherein the composition comprises 0.005 to 5 mass % modified-TiO₂ nanoparticles and at least 20 mass % polymers.

14. The polymeric composition of claim 8 wherein the composition comprises 0.05 to 0.1 mass % modified-TiO₂ nanoparticles.

15. The polymeric composition of claim 8 wherein the modified-TiO₂ nanoparticles are present in an amount effective to improve degree of vinyl conversion of the polymer precursors under light irradiation.

16. The polymeric composition of claim 8 comprising the solvent wherein the solvent has a boiling point below 159° C.

17. The polymeric composition of claim 16 wherein the solvent is one or more selected from the group consisting of water, ethanol, methanol, toluene, ethyl ether, cyclohexane, iso-propanol, chloroform, ethyl acetate hexane, and heptanes.

18. The polymeric composition of claim 8 comprising the solvent wherein the modified-TiO₂ nanoparticles and solvent form an organosol or a colloidal dispersion.

19. The polymeric composition of claim 8 wherein the polymer precursor is selected from the group consisting of acrylates, methacrylates and dimethacrylates.

20. The polymeric composition of claim 19 wherein the polymer precursor is at least one selected from the group consisting of ethylenedimethacrylate (“EDMA”), bisphenol A glycidyl methacrylate (“BisGMA”), triethyleneglycol dimethacrylate (“TEGDMA”), 1,6-bis(methacryloxy-2-ethoxycarbonylamino)-2,4,4-trimethylhexane (UDMA), pyromellitic glycerol dimethacrylate (PMGDM), and 2-hydroxyethyl methacrylate (HEMA).

21. The polymeric composition of claim 8 wherein the polymer precursor is formed from one or more derivatives selected from the group consisting of ether, carbonate, silicone, olefin, urethane, styrene, vinylaromatic, amide, imide, vinylhalide, phenylene oxide, and ketone.

22. The polymeric composition of claim 8 wherein the composition has a controllable hydrophilicity, wherein the hydrophilicity is determined according to water contact angle measured by goniometer, and a larger water contact angle means lesser hydrophilicity.

23. The polymeric composition of claim 8 wherein the hydrophilicity is controlled by the polymer precursor, mass fraction of modified-TiO₂, and intensity and duration of UV or light irradiation.

24. The polymeric composition of claim 8 wherein the modified-TiO₂ nanoparticles comprises >60% anatase and <40% rutile.

25. The polymeric composition of claim 8 wherein the polymeric composition is a dental composition selected from the group consisting of a dental adhesive, a dental sealant, and a dental composite.

26. The polymeric composition of claim 8 wherein the polymeric composition is a synthetic rubber, epoxy fiber glass, paint material, coating materials, anti-microbial packing material, scratch resistant material, or self-clean material.

27. A process of preparing a polymeric composition comprising mixing modified-TiO₂ nanoparticles, a solvent, and polymer precursors to form a colloidal dispersion or organosol; wherein the modified-TiO₂ nanoparticles are prepared by modifying titanium dioxide nanoparticles with at least one short-chain unsaturated organic compound comprising 2 to 10 carbon atoms with or without branch chains, wherein after modification, the mass fraction of organic components in the modified-TiO₂ nanoparticles is in the range from 0.5% to 10% determined by thermal gravimetric analysis.

28. The process of claim 26 wherein the colloidal dispersion or organosol is formed by centrifuging the mixture of modified-TiO₂ nanoparticles and the solvent.

29. The process of claim 27 further comprising centrifuging the modified-TiO₂ nanoparticles and the solvent at a spin rate in a range from 1000 rpm to 5000 rpm for 1 min to 10 min.

30. The process of claim 26 wherein the solvent is water, one or more organic solvent(s) with a boiling point below 159° C., or mixtures thereof

31. The process of claim 26 further comprising removing the solvent by vacuum or by air blowing.

32. The process of claim 30 further comprising, after removing the solvent, irradiating the mixture of modified-TiO₂ nanoparticles and polymer precursors with light to form a polymer containing modified-TiO₂ nanoparticles.

33. The process of claim 31 wherein the mixture is irradiated with blue light with wavelength between 350 nm to 550 nm

34. The process of claim 31 wherein the modified-TiO₂ nanoparticles are present in an amount effective as an initiator or co-initiator of photo-polymerization to improve the degree of vinyl conversion of polymer precursors during the irradiation with light.

35. The process of claim 31 wherein the modified-TiO₂ nanoparticles modify the hydrophilicity of the polymer composition after light irradiation.

36. The process of claim 26 wherein the dental composition comprises 0.05 to less than 1% mass fraction of modified-TiO₂ nanoparticles.

37. The process of claim 26 further comprising controlling the hydrophilicity of the polymer composition, wherein the hydrophilicity is determined according to water contact angle measured by goniometer.

38. The process of claim 36 further comprising controlling the hydrophilicity by adjusting the polymer precursor, mass fraction of modified-TiO₂, and intensity and duration of UV or light irradiation.

39. A polymeric composition comprising an irradiated mixture of modified-TiO₂ nanoparticles and polymer precursors; wherein the modified-TiO₂ nanoparticles comprise titanium dioxide nanoparticles modified with at least one short-chain unsaturated organic compound comprising 2 to 10 carbon atoms with or without branch chains; wherein the mass fraction of organic components in the modified-TiO₂ nanoparticles is in the range from 0.5% to 10% determined by thermal gravimetric analysis.

40. The polymeric composition according to claim 39 wherein the irradiated mixture is a light irradiated mixture.

41. The polymeric composition according to claim 39 wherein the polymeric composition comprises thermoplastic, thermoset, or cross-linked polymeric materials.

42. The polymeric composition according to claim 41 wherein the polymeric materials are selected from the group consisting of acrylate resins, methacrylate resins, and dimethacrylate esters resins, epoxy resins, polycarbonate, silicone, polyester, polyether, polyolefin, synthetic rubber, polyurethane, nylon, polystyrene, polyvinylaromatic, polyamide, polyimide, polyvinylhalide, polyphenylene oxide, polyketone, and copolymers and blends thereof

43. The polymeric composition of claim 39 wherein the composition comprises 0.005 to 5 mass % modified-TiO₂ nanoparticles and at least 20 mass % polymers.

44. The polymeric composition of claim 39 wherein the polymeric composition is a dental composition selected from the group consisting of a dental adhesive, a dental sealant, and a dental composite.

45. The polymeric composition of claim 39 wherein the polymeric composition is a synthetic rubber, epoxy fiber glass, paint material, coating materials, anti-microbial packing material, scratch resistant material, or a self-clean material.

46. The polymeric composition of claim 39 wherein the modified-TiO₂ nanoparticles are effective to eliminate bacteria in the polymeric composition.

47. The polymeric composition of claim 39 wherein the polymeric composition containing modified-TiO₂ nanoparticles has a higher modulus and a higher hardness over the same polymeric composition containing unmodified-TiO₂ nanoparticles.

48. The polymeric composition of claim 39 wherein the polymeric composition containing modified-TiO₂ nanoparticles has a higher modulus and a higher hardness over the same polymeric composition containing no TiO₂ nanoparticles.

49. A dental adhesive comprising 0.05 to 1 mass % modified-TiO₂ nanoparticles, polymer precursors, and optionally, inorganic fillers;
wherein the modified-TiO₂ nanoparticles comprise titanium dioxide nanoparticles modified with at least one short-chain unsaturated organic compound comprising 2 to 10 carbon atoms with or without branch chains;
wherein the mass fraction of organic components in the modified-TiO₂ nanoparticles is in the range from 0.5% to 10% determined by thermal gravimetric analysis; and wherein the modified-TiO₂ nanoparticles have a particle size greater than 1 nm and less than 100 nm; and
wherein the polymer precursor is at least one selected from the group consisting of ethylenedimethacrylate (“EDMA”), bisphenol A glycidyl methacrylate (“BisGMA”), triethyleneglycol dimethacrylate (“TEGDMA”), 1,6-bis(methacryloxy-2-ethoxycarbonylamino)-2,4,4-trimethylhexane (UDMA), pyromellitic glycerol dimethacrylate (PMGDM), and 2-hydroxyethyl methacrylate (HEMA); or
wherein the polymer precursor is formed from one or more derivative selected from the group consisting of carboxylic acid, ether, carbonate, silicone, olefin, urethane, styrene, vinylaromatic, amide, imide, vinylhalide, phenylene oxide, and ketone.
